(19) United States
(12) Patent Application Publication
(10) Pub. No.: US 2023/0218418 A1
(43) Pub. Date: Jul. 13, 2023

(54) SKELETAL STRUCTURE CORRECTION APPARATUS AND METHOD FOR ASSESSING RISK TO MUSCULOSKELETAL SYSTEM OF WORKERS

(71) Applicant: Electronics and Telecommunications Research Institute, Daejeon (KR)

(72) Inventors: Youngjin KWON, Daejeon (KR); Do Hyun KIM, Daejeon (KR)

(73) Assignee: Electronics and Telecommunications Research Institute, Daejeon (KR)

(21) Appl. No.: 18/083,151

(22) Filed: Dec. 16, 2022

(30) Foreign Application Priority Data
Jan. 7, 2022 (KR) 10-2022-0002811

(51) Int. Cl. A61F 5/01; A61B 5/11; G16H 50/30

(52) U.S. Cl. CPC A61F 5/0102; A61B 5/1116; G16H 50/30

(57) ABSTRACT

Provided are a skeletal structure correction apparatus and a skeletal structure correction method for assessing risks to a musculoskeletal system of a worker. The skeletal structure correction method quantitatively determines an analysis time and uncertain body motions by providing an assessment tool for significantly reducing the workload of ergonomic/industrial engineering experts investigating the risks to the musculoskeletal system.

### CROSS-REFERENCE TO RELATED APPLICATION

This application claims the benefit of Korean Patent Application No. 10-2022-0002811 filed on Jan. 7, 2022, in the Korean Intellectual Property Office, the entire disclosure of which is incorporated herein by reference for all purposes.

### BACKGROUND

### 1. Field of the Invention

One or more embodiments relate to a skeletal structure correction apparatus and method for investigating risks to a musculoskeletal system of a worker, and more specifically, to an apparatus and method for assisting an assessment opinion of an existing expert by automating a process of investigating the risks to the musculoskeletal system using a deep learning model.

### 2. Description of the Related Art

Recently, as work-related musculoskeletal diseases among workers increase across all industries, they have caused great economic loss and loss in terms of the health of workers. The main causes of these musculoskeletal diseases are known to be risks including repetitive motions which may cause musculoskeletal diseases, inappropriate postures (unnatural or difficult operation postures), excessive force, contact stress (body contact with sharp surfaces), and vibration. By removing or reducing risks causing musculoskeletal diseases in accordance with safety and health rules to prevent musculoskeletal diseases, it is possible to improve or help the operation environment of workers.

The investigation period of risks related to musculoskeletal diseases should be regularly conducted every 3 years after the initial investigation of risks, and the working conditions, such as workload, posture, time, and method, should be improved according to the investigation results. Currently, for the investigation method of risks related to musculoskeletal diseases, a health manager of a workplace employed by the employer or a specialist in a specialized institution is requested to perform the investigation of the risks.

Risk investigation experts divide the classification of operations by each unit operation, if it is possible to divide the operations into unit operations according to the operation of the worker. In order to analyze the divided operations, images of the operation are obtained in angles necessary for the analysis, such as the whole body, upper body, lower body, feet, and hands, by using a camera, and the image is played to extract the frames; the frames are analyzed in detail by motion or angles based on the judgment standard of the experts.

However, in order to assess the inappropriate operation posture of workers which cause musculoskeletal diseases, risk investigation experts also need to analyze several hours of operation images, and the experts may have different interpretations according to the angle of the obtained images.

### SUMMARY

Embodiments provide an apparatus and method for correcting skeletal information of a worker according to an action level of an operation posture of the worker, by extracting a posture of the worker from an operation image obtained by using deep learning technology.

Embodiments provide an apparatus and method for proposing a section about an operation image of a posture corresponding to a threshold predetermined by experts without monitoring all images, to experts investigating risks to a musculoskeletal system of workers.

According to an aspect, there is provided a skeletal structure correction method including collecting an operation image of a worker, divided into operation units, determining skeletal information of the worker by considering a capturing angle of the collected operation image, determining an action level of an operation posture of the worker for each operation unit based on the determined skeletal information of the worker, and correcting the skeletal information of the worker, corresponding to the determined action level.

The collecting of the operation image of the worker may include collecting an operation image including the whole body of the worker or collecting a plurality of operation images of a same operation captured in different angles, through an image device.

The collecting of the operation image of the worker may include collecting an operation image divided into at least one operation unit of a waist, upper limbs, and lower limbs, according to an operation motion of the worker.

The determining of the skeletal information of the worker may include determining the skeletal information of the worker by using at least one operation image of a waist, upper limbs, and lower limbs according to an operation posture of the worker.

The determining of the skeletal information of the worker may include estimating a body part of the worker corresponding to the operation unit, determining a location of a joint of the worker by considering a feature of a musculoskeletal system according to the body part of the worker, and determining skeletal information of a worker corresponding to the location of the joint of the worker.

The determining of the action level may include determining an action level indicating a risk level of an operation for each operation posture of the worker, by considering a motion direction of the joint according to the location of the joint.

The correcting of the skeletal information of the worker may include correcting the skeletal information of the worker to improve the operation posture of the worker corresponding to the action level.

According to an aspect, there is provided a skeletal structure correction method including displaying operation images including skeletal information about an operation posture of a worker working in a workplace, activating and displaying at least one operation image including an operation posture corresponding to a predetermined threshold among the operation images, and displaying a correction result of the skeletal information of the worker within the activated and displayed operation image on a screen, by considering an angle of a joint according to the operation posture.

The displaying of the operation images may include displaying, on the screen, an operation image divided into at least one operation unit of a waist, upper limbs, and lower limbs, according to an operation motion of the worker.

The activating and displaying of the operation image may include determining whether each angle of the joint according to the operation posture of the worker exceeds a predetermined threshold in the operation images and activating and displaying at least one operation image exceeding the predetermined threshold.

The displaying of the correction result of the skeletal information on the screen may include correcting the skeletal information of the worker to improve the operation posture of the worker by considering a feature of a musculoskeletal system, according to a body part of the worker and displaying the corrected skeletal information of the worker on the screen.

According to an aspect, there is provided a skeletal structure correction apparatus including a processor. The processor may be configured to collect an operation image of a worker, divided into operation units, determine skeletal information of the worker by considering a capturing angle of the collected operation image, determine an action level of an operation posture of the worker for each operation unit based on the determined skeletal information of the worker, and correct the skeletal information of the worker, corresponding to the determined action level.

The processor may be configured to estimate a body part of the worker corresponding to the operation unit, determine a location of a joint of the worker by considering a feature of a musculoskeletal system according to the body part of the worker, and determine skeletal information of a worker corresponding to the location of the joint of the worker.

The processor may be configured to determine an action level indicating a risk level of an operation for each operation posture of the worker, by considering a motion direction of the joint according to the location of the joint.

The processor may be configured to determine the action level by considering an angle according to a location of a joint of the worker within the operation image.

The processor may be configured to correct the skeletal information of the worker to improve the operation posture of the worker corresponding to the action level.

According to an aspect, there is provided a skeletal structure correction apparatus including a processor. The processor may be configured to display operation images including skeletal information about an operation posture of a worker working in a workplace, activate and display at least one operation image including an operation posture corresponding to a predetermined threshold among the operation images, and display a correction result of the skeletal information of the worker within the activated and displayed at least one operation image on a screen, by considering an angle of a joint according to the operation posture.

The activating and displaying of the operation image may include determining whether each angle of the joint according to the operation posture of the worker exceeds a predetermined threshold in the operation images and activating and displaying at least one operation image exceeding the predetermined threshold

The displaying of the correction result of the skeletal information on the screen may include correcting the skeletal information of the worker to improve the operation posture of the worker by considering a feature of a musculoskeletal system, according to a body part of the worker and displaying the corrected skeletal information of the worker on the screen.

Additional aspects of embodiments will be set forth in part in the description which follows and, in part, will be apparent from the description, or may be learned by practice of the disclosure.

According to embodiments, a skeletal structure correction apparatus may extract a posture of a worker from an obtained operation image by using deep learning technology to correct skeletal information of the worker according to an action level of an operation posture of the worker.

According to embodiments, the skeletal structure correction apparatus may analyze an operation relatively effectively by providing experts investigating risks to a musculoskeletal system of the worker with a section of an operation image of a posture corresponding to a threshold predetermined by the experts, among all operation images.

According to embodiments, the skeletal structure correction apparatus may provide an interface for significantly reducing workload of ergonomic or industrial engineering experts investigating the risks to the musculoskeletal system, to quantitatively determine analysis time and uncertain body motions.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a diagram illustrating an image device and a skeletal structure correction apparatus for correcting skeletal information of a worker according to an embodiment;

FIG. 2 is a diagram illustrating a detailed configuration of a skeletal structure correction apparatus according to an embodiment;

FIG. 3 is a diagram illustrating a process of reconfiguring a three-dimensional (3D) human body model by extracting an operation posture of a worker from an operation image according to an embodiment;

FIG. 4 is a diagram illustrating a process of generating skeletal information of a worker according to a capturing angle of an operation image according to an embodiment;

FIG. 5 is a diagram illustrating an interface for correcting a skeletal information of a worker according to an embodiment;

FIG. 6 is a diagram illustrating a reference model for calculating an angle of a major skeletal structure from among skeletal information of a worker according to an embodiment;

FIG. 7 is a diagram illustrating a process of determining skeletal information of a worker by considering an occluded area in an operation image according to an embodiment;

FIG. 8 is a flowchart illustrating a skeletal structure correction method according to an embodiment; and

FIG. 9 is a flowchart illustrating a skeletal structure correction method according to an embodiment.

### DETAILED DESCRIPTION

Hereinafter, embodiments will be described in detail with reference to the accompanying drawings.

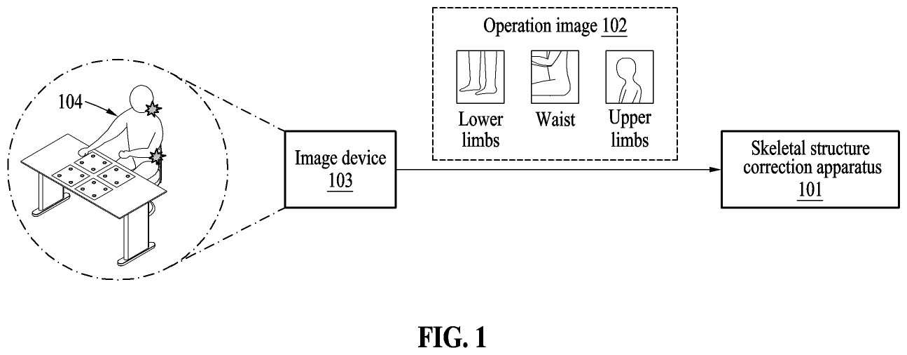

FIG. 1 is a diagram illustrating an image device and a skeletal structure correction apparatus for correcting skeletal information of a worker according to an embodiment.

Referring to FIG. 1, a skeletal structure correction apparatus 101 may collect an operation image 102 of a worker at work from an image device 103. Here, the image device 103 may be a device installed at various angles to assess the risks to the musculoskeletal system of a worker 104 in the workplace. For example, the image device 103 may be a camera which collects at least one of a color image and a depth image to observe the movement of the worker 104.

Specifically, the skeletal structure correction apparatus 101 may collect the operation image 102 of the worker 104 divided into operation units to determine the skeletal information of the worker 104 by considering the capturing angle of the operation image 102. The operation image 102 is an image of at least one body part among the waist and the upper and lower limbs taken at different angles of the same operation, depending on the angle. As an example, the operation image 102 may include an operation image of the upper limb from the left side, an operation image of the upper limb from the right side, an operation image of the lower limb from the left side, an operation image of the lower limb from the right side, and a detailed operation image of the hand of the upper limb from the right side, corresponding to each of a front, a side, and a plane.

Here, an operation unit may be a reference for dividing each operation posture of the motion performed by a worker in the operation environment into the waist, the upper limb, and the lower limb. The operation environment may be a condition for defining work efficiency of each work process in which at least one of equipment, workload, and work speed in the workplace is reflected. For example, the operation environment may include conditions reflecting an ergonomic design, such as operation height, tools suitable for the job, auxiliary devices, operation space, and free space, within the workplace. The operation posture may refer to a motion which the worker needs to take as a human operation, according to the features of the operation process. For example, the operation posture may be a motion of lifting or pushing an object, and a movement of the body or the limbs to perform operations such as the height of the elbow and the height between the knees and the shoulders.

The skeletal structure correction apparatus 101 may predict the body part of the worker 104 according to the operation unit and determine the location of the joint of the worker by considering the features of the musculoskeletal system according to the predicted body part of the worker 104. The skeletal structure correction apparatus 101 may determine skeletal information of the worker corresponding to the location of the joint of the worker.

The skeletal structure correction apparatus 101 may determine an action level of the operation posture of a worker for each operation unit based on the determined skeletal information of the worker. The skeletal structure correction apparatus 101 may determine the action level indicating a risk level of the operation for each operation posture of the worker, by considering the motion direction or angle of the joint according to the location of the joint. The skeletal structure correction apparatus 101 may correct skeletal information of the worker corresponding to the action level.

In addition, the skeletal structure correction apparatus 101 may display the corrected skeletal information of the worker 104 on the screen of an interface in order to assist the assessment opinion of the experts conducting the risk investigation.

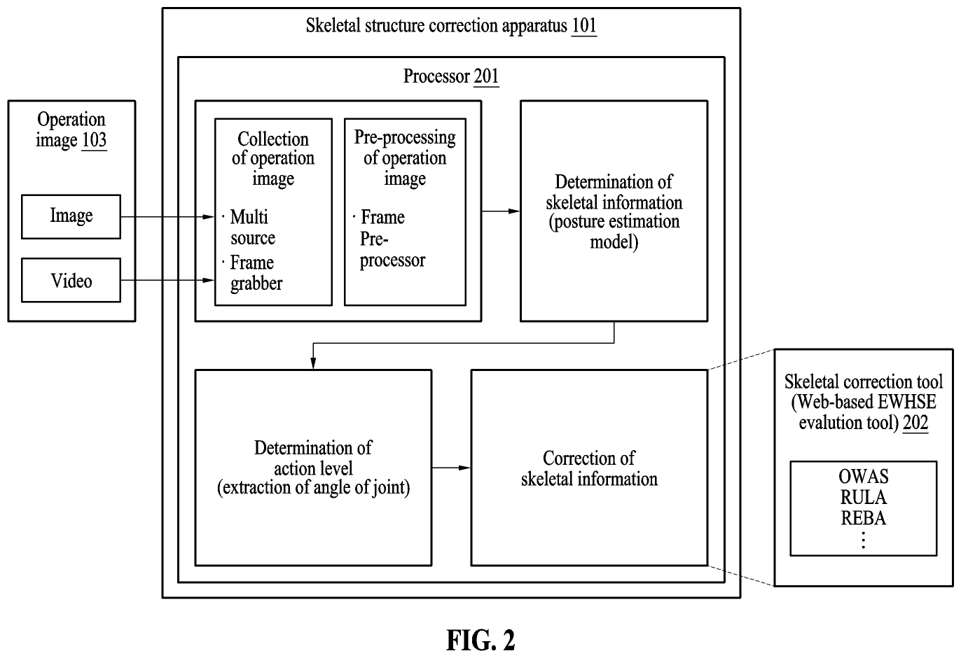

FIG. 2 is a diagram illustrating a detailed configuration of a skeletal structure correction apparatus according to an embodiment.

Referring to FIG. 2, the skeletal structure correction apparatus 101 may include a processor 201, and the processor 201 may collect the operation image 102 of a worker provided locally or remotely within the workplace. The operation image 102 may include a color image or a depth image depending on the type of image device, and a color image may be collected in the form of an image or a video.

When the video form is collected, the skeletal structure correction apparatus 101 may use the operation image 102 that has been pre-processed in the unit of frames. That is, the skeletal structure correction apparatus 101 may be use the operation image 102 collected in the unit of frames as an input to determine the skeletal information, after performing primary processing of the operation image 102 with a correction, such as size correction or rotation.

The skeletal structure correction apparatus 101 may determine the skeletal information of the worker according to the capturing angle of the operation image 102. In this case, the skeletal structure correction apparatus 101 may apply a posture estimation model to estimate the body part of the worker and then determine the location of the joint of the body part. The body part may be a part corresponding to the waist and the upper and lower limbs for each operation unit, and the location of the joint may be an area in which the bones are connected for each body part.

The skeletal structure correction apparatus 101 may determine the action level of the operation posture of the worker for each operation unit based on the skeletal information of the worker. The skeletal structure correction apparatus 101 may calculate an angle of the joint using the skeletal information.

In detail, the joint may be classified into a nonaxial joint, a uniaxial joint, a biaxial joint, and a triaxial joint, and each axial joint may be classified as follows.

\textcircled{1} A nonaxial joint may include a joint without a rotation axis, a gliding joint, and a plane joint. A representative joint of the gliding joint may include a joint between the wrist bone and the ankle bone, and a representative joint of the plane joint may include an acromioclavicular joint (the plane joint between the acromion of the scapula and the distal clavicle).

\textcircled{2} A uniaxial joint may be a joint that moves around one motion axis and is capable of one type of rotational movement, and may include a hinge joint and a pivot joint. A representative joint of the hinge joint may include an elbow (elbow joint), a knee joint (knee joint), and an interphalangeal joint of a finger. In addition, a representative joint of the pivot joint may include the neck, the cervical spine, the forearm, the shinbone, and the calf bone of the leg.

\textcircled{3} A biaxial joint may be a joint that moves around two axes and a joint capable of two types of rotational movements: flexion and extension, and abduction and adduction, and may include an ellipsoidal joint and a saddle joint. A representative joint of the ellipsoidal joint may include a wrist joint and a metacarpophalangeal joint of the finger. In addition, a representative joint of the saddle joint may include a wrist and thumb.

\textcircled{4} A triaxial joint is a joint that moves around three axes as an axial joint and may include a ball and socket joint. A representative joint of the ball and socket joint may include the shoulder and buttocks.

The skeletal structure correction apparatus 101 may calculate the angle of the joint considering the motion direction of each joint of each location of the joint. Here, the motion direction of the joint may represent the amount of motion, the degree of motion freedom, and the range of motion according to the axis of the joint and the rotational movement.

The skeletal structure correction apparatus 101 may determine the action level of the corresponding task by using the rules of the ergonomic assessment tools according to the calculated angle of the joint. The action level is called an action category, and may be a value which comprehensively determines a score coded by the ergonomic posture assessment tool and a value indicating the level of action necessary for the current operation. Here, the value of the action level varies depending on the assessment tool, and a higher number may indicate that the operation needs more action. The skeletal structure correction apparatus 101 may determine the action level of the operation by using the assessment tool. The assessment tool is a tool for assessing the level of ergonomic operations, and may include tools, such as Ovako Working posture Analysis

System (OWAS)/Rapid Upper Limb Assessment (RULA)/Rapid Entire Body Assessment (REBA). For example, the skeletal structure correction apparatus 101 may determine the action level by using a table of each assessment tool for body part scores according to different angles.

The skeletal structure correction apparatus 101 may use deep learning technology to estimate the posture of the worker 104 based on the operation image 102 to classify the capturing angle of the operation image 102 to find a body part with high reliability. The skeletal structure correction apparatus 101 may determine the overall posture of the worker 104 according to the capturing angle of the operation image 102.

According to the present invention, there may be restrictions on the quality (occlusion) of the operation image 102 collected according to the operation environment of a worker. Accordingly, the skeletal structure correction apparatus 101 may determine the overall posture of the worker 104 by matching the operation images 102 taken at different angles, to extract an operation image of a body part suitable for assessing the risks to the musculoskeletal system of the worker 104 from the operation image 102 taken at different angles of the same operation, by using deep learning technology.

The skeletal structure correction apparatus 101 may correct skeletal information of the worker corresponding to the action level. The skeletal structure correction apparatus 101 may correct the skeletal information of the worker by using an assessment tool using a skeletal correction tool 202.

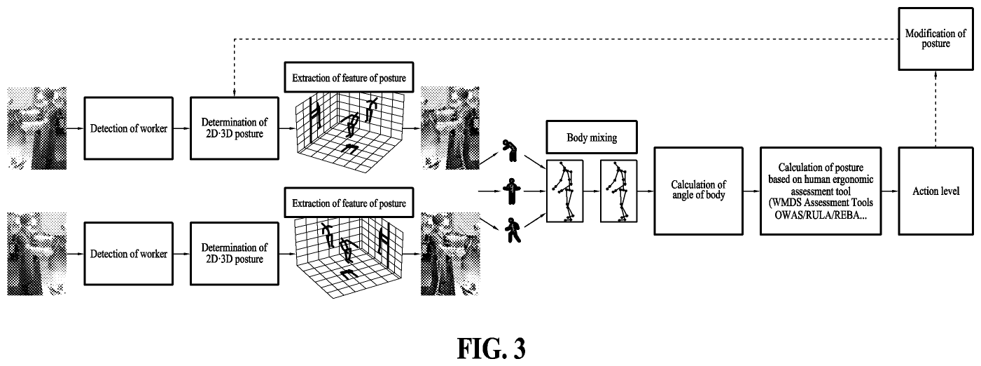

FIG. 3 is a diagram illustrating a process of reconfiguring a three-dimensional (3D) human body model by extracting the operation posture of a worker from an operation image according to an embodiment.

Referring to FIG. 3, a skeletal structure correction apparatus may determine the skeletal information of the worker from the operation image by using a posture estimation model. The skeletal information may be information indicating a pose or an operation posture of the worker. To this end, the skeletal structure correction apparatus may configure a network (Human Detecotor/postureNet) for extracting skeletal information.

The skeletal structure correction apparatus may determine the position of the worker in the operation image. The skeletal structure correction apparatus may reconstruct either a two-dimensional (2D) or a 3D human body model in order to estimate the posture of the worker based on the determined position of the worker.

The skeletal structure correction apparatus may estimate the posture of the worker through a network-based 2D human body model or a 3D human body model. The skeletal structure correction apparatus may calculate the capturing angle of the operation image collected by an imaging apparatus according to the estimated posture of the worker. In detail, the skeletal structure correction apparatus may estimate a posture corresponding to the front, side, and plane of the worker in the operation image based on the network. Here, the estimated posture may be a posture used to limit a specific part to determine the risk to the musculoskeletal system among body parts of the worker.

The skeletal structure correction apparatus may calculate the capturing angle of the operation image, such as the upper limb/the lower limb/the arm/the waist/the side/the front, from the estimated posture and the operation image. In addition, the skeletal structure correction apparatus may determine which part of the body parts of the worker corresponding to the operation posture the operation image is captured to determine, corresponding to the calculated capturing angle. The skeletal structure correction apparatus may determine the action level according to the assessment tool, based on the determined angle of the joint in the operation image.

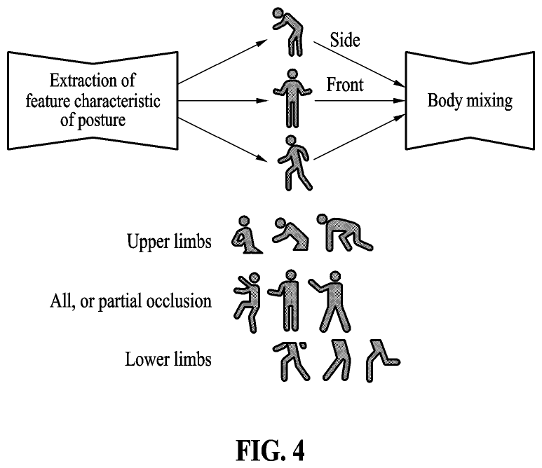

FIG. 4 is a diagram illustrating a process of generating skeletal information of a worker according to a capturing angle of an operation image according to an embodiment.

Referring to FIG. 4, a skeletal structure correction apparatus may collect operation images of various angles and angles of view so that an expert may more precisely analyze the operation posture of the worker according to unit operation. The skeletal structure correction apparatus may collect operation images corresponding to the front, side, and plane according to the working motion of the worker. The skeletal structure correction apparatus may distinguish whether the body part of the worker included in the operation image is the upper limb, the lower limb, or the entire area (or partial occlusion) corresponding to the front, side, and plane, respectively. In other words, the skeletal structure correction apparatus may determine which state of the worker of the capturing angle the collected operation image represents.

The skeletal structure correction apparatus may estimate the posture corresponding to the front, side, and plane of the worker in the operation image. The skeletal structure correction apparatus may estimate a posture used to limit a specific part to determine the risk to the musculoskeletal system among the body parts of the worker, according to the operation image.

The skeletal structure correction apparatus may use the operation image including the estimated posture to comprehensively determine whether the worker is working with the risks to the musculoskeletal system. The skeletal structure correction apparatus may correct the skeletal information of the worker by performing an operation unit through the body synthesis network, by considering the angle according to the estimated posture.

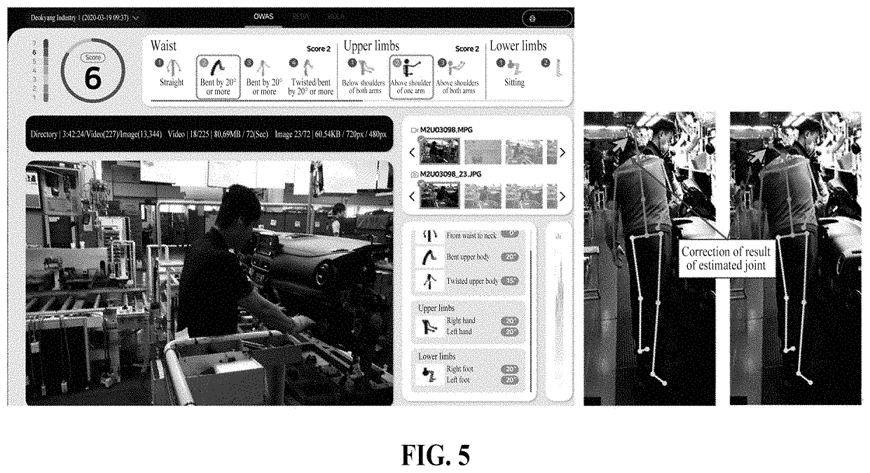

FIG. 5 is a diagram illustrating an interface for correcting the skeletal information of a worker according to an embodiment.

Referring to FIG. 5, a skeletal structure correction apparatus may provide an interface for correcting the skeletal information of the worker. The skeletal structure correction apparatus may display operation images including the skeletal information of the operation posture of the worker who works in a workplace, through an interface.

The skeletal structure correction apparatus may activate and display at least one operation image including an operation posture corresponding to a predetermined threshold among operation images. The skeletal structure correction apparatus may determine whether each angle of the joint according to the operation posture of the worker among the operation images exceeds the predetermined threshold. The skeletal structure correction apparatus may activate and display at least one operation image exceeding a threshold value.

Here, the skeletal structure correction apparatus may calculate the angle of the worker's major joints according to the operation image and generate basic data for the determination, according to the rules of the ergonomic assessment tools of harmful operation. To this end, the skeletal structure correction apparatus may express the analyzed information on a user interface (UI) of FIG. 5 to express a frame which exceeds the threshold value according to predetermined condition (angle level of each joint) of the expert. The skeletal structure correction apparatus may provide the interface so that the expert may intensively analyze the image of the corresponding part using the pre-processed result.

The skeletal structure correction apparatus may display, on the screen, a result of correcting the skeletal information of the worker in the activated and displayed operation image by considering the angle of the joint of the worker according to the operation posture.

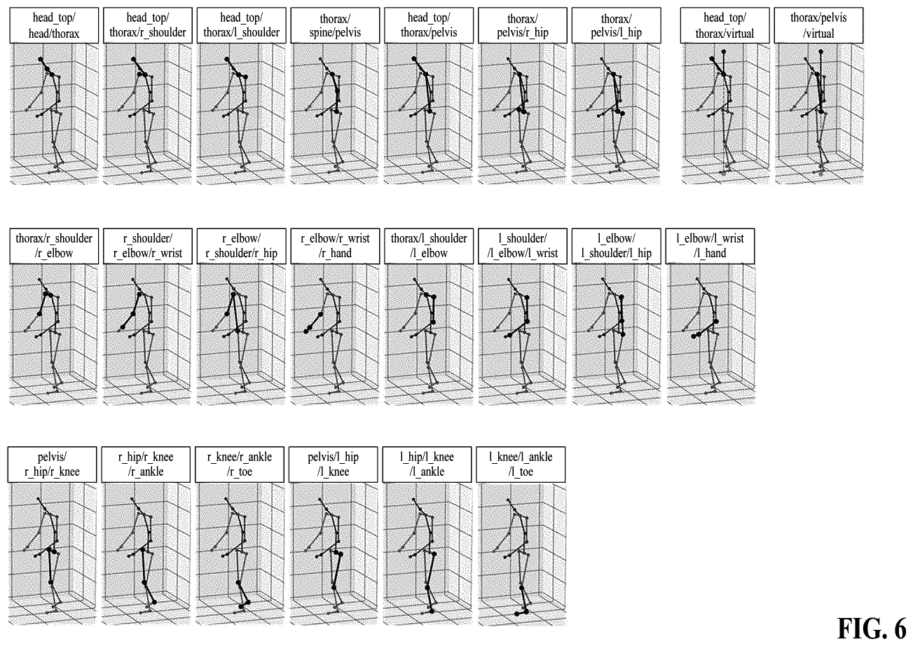

FIG. 6 is a diagram illustrating a reference model for calculating an angle of a major skeletal structure from among skeletal information of a worker according to an embodiment; Referring to FIG. 6, according to the skeletal structure correction apparatus may match a combination of mutually related joints by operation unit in various views. the skeletal structure correction apparatus may perform an operation unit of a minimum unit of a meaningful process, which is a repetitive operation divided in the entire process.

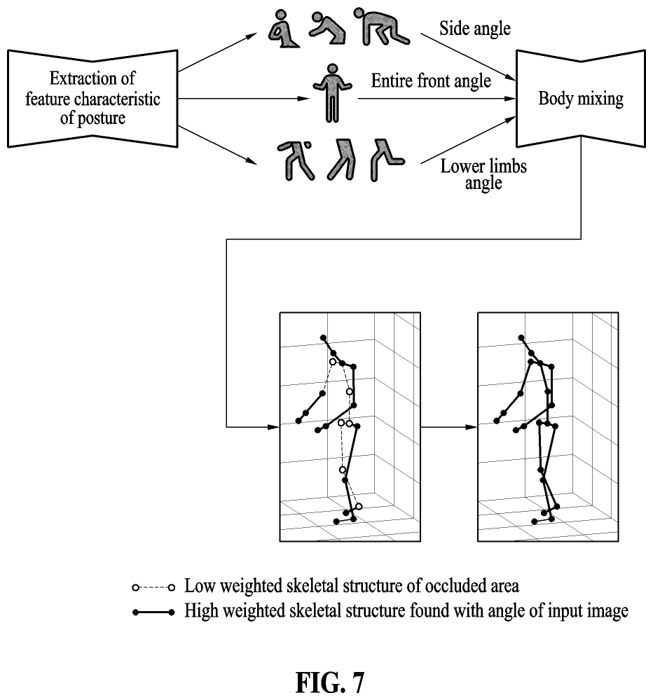

FIG. 7 is a diagram illustrating a process of determining the skeletal information of the worker by considering an occluded area in the operation image according to an embodiment.

Referring to FIG. 7, according to the present disclosure, an issue in the operation space may occur in that the input of the operation image may not be taken at a standardized angle. In this case, it may be difficult for the expert to make an analysis only with a standardized operation image with the present disclosure.

Accordingly, the skeletal structure correction apparatus may enable the experts to finally determine the skeletal information of the worker by combining the joints related to each other by matching the estimated values of the bodies, occluded due to the angle or by instruments, with the posture of the worker by operation unit in the operation image, while reflecting the previous capturing methods. The skeletal structure correction apparatus may capture the operation unit with two or more image devices (e.g., a camera) from various angles. Here, the image device may perform time synchronization between devices through a separate server coupled with the image device or perform time synchronization by using a separate recording program. In addition, a separate image acquisition system may synchronize the time of the operation image by using a self-generated synchronization signal. The synchronization signal may utilize the reference clock of the H/W interface or the S/W-like Timer Tick information in the program.

Accordingly, the skeletal structure correction apparatus may be used to match the posture information of the occluded part using the operation image described above, in which time synchronization between the cameras is performed.

To match the posture information, the skeletal structure correction apparatus may use the always visible 1) length of the joints of the head-neck as a reference value for the scaler, and 2) length of the representative joints of the body, neck-pelvis, 3) length of the joints of the neck-shoulder, and 4) length of the joints of the pelvis-hip as an auxiliary value. Both shoulders and hips, which are left/right paired joints, may be used to match information of the posture estimated from different scaled views.

Operation images acquired with multiple image devices cannot be acquired by accurately fixing the view angle/distance with the subject in the field, unless acquired in a studio, so a scaler which scales using the representative joints of the body may be required. In addition, the skeletal structure correction apparatus may mix the body parts of the worker by using auxiliary joints to synthesize the information of the scaled joints.

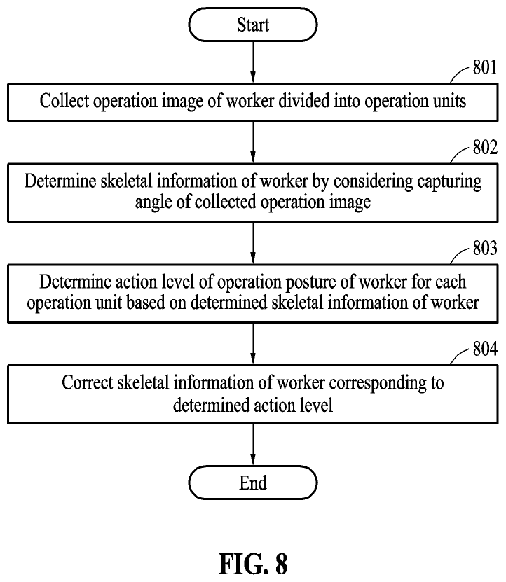

FIG. 8 is a flowchart illustrating a skeletal structure correction method according to an embodiment.

In operation 801, the skeletal structure correction apparatus may collect the operation image of the worker divided into operation units. The skeletal structure correction apparatus may collect a plurality of operation images of the same operation captured at different angles, performed by the worker, through an image device. The skeletal structure correction apparatus may collect operation images of different angles subdivided into a waist, upper limbs, and lower limbs, according to operation units corresponding to the same operation.

In operation 802, the skeletal structure correction apparatus may determine the skeletal information of the worker by considering the capturing angle of the operation image. The skeletal structure correction apparatus may determine the skeletal information of the worker by using the operation image of at least one of the waist, upper limbs, and lower limbs according to the operation posture of the worker.

The skeletal structure correction apparatus may predict a body part of the worker corresponding to the operation unit. For example, when the operation unit corresponds to the upper limb, the skeletal structure correction apparatus may determine the body part of the worker to include at least one of a finger, wrist, neck, shoulder, and elbow. In addition, when the operation unit corresponds to the lower limb, the skeletal structure correction apparatus may determine the body part of the worker to include at least one of buttocks, knee, ankle, and foot.

The skeletal structure correction apparatus may determine the location of the joint of the worker according to the type of the axis of the joint and the rotational movement. The skeletal structure correction apparatus may determine the skeletal information of the worker corresponding to the location of the joint of the worker.

In operation 803, the skeletal structure correction apparatus may determine the action level of the operation posture of the worker for each operation unit based on the skeletal information of the worker. Here, the action level may be a value indicating the level at which the current task requires an action by comprehensively determining the score coded by the ergonomic posture assessment tool. The skeletal structure correction apparatus may determine the action level indicating a risk level of an operation for each operation posture of the worker, by considering a motion direction of the joint according to the location of the joint.

In addition, the skeletal structure correction apparatus may consider the body part exposed to occupational musculoskeletal disease according to the operation type of the worker and the status of each body part. That is, the skeletal structure correction apparatus may determine the action level of the operation posture of the worker by considering the symptoms or frequency of musculoskeletal diseases to which the workers are exposed due to operations, such as farming and manufacturing, related to the worker's job.

In operation 804, the skeletal structure correction apparatus may correct the skeletal information of the worker corresponding to the action level. The skeletal structure correction apparatus may correct the skeletal information of the worker to improve the operation posture of the worker in response to the action level.

The skeletal structure correction apparatus may extract an operation image including skeletal information exceeding the threshold value corresponding to the predetermined condition of the expert, based on the action level. The skeletal structure correction apparatus may correct the skeletal information of the worker in the extracted operation image to correspond to the angle level of each joint.

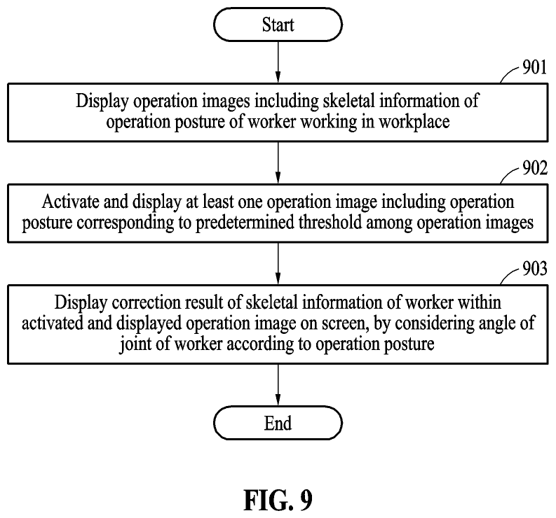

FIG. 9 is a flowchart illustrating a skeletal structure correction method according to an embodiment.

In operation 901, the skeletal structure correction apparatus may display operation images including skeletal information of the operation posture of the worker working in the workplace. The skeletal structure correction apparatus may display, on the screen, an operation image divided into at least one operation unit of a waist, upper limbs, and lower limbs according to an operation motion of the worker. Here, the skeletal structure correction apparatus may divide the operation image, in which primary processing is completed, into each operation unit through a pre-processing process, such as size/rotation, and display the divided operation image on the screen.

In operation 902, the skeletal structure correction apparatus may activate and display at least one operation image including an operation posture corresponding to a predetermined threshold among the operation images. The skeletal structure correction apparatus may determine whether each angle of the joint according to the operation posture of the worker among the operation images exceeds the predetermined threshold. The skeletal structure correction apparatus may activate and display at least one operation image exceeding the predetermined threshold value.

In operation 903, the skeletal structure correction apparatus may display a correction result of the skeletal information of the worker within the activated and displayed at least one operation image on the screen, by considering the angle of the joint according to the operation posture. The skeletal structure correction apparatus may correct the skeletal information of the worker to improve the operation posture of the worker by considering a feature of a musculoskeletal system, according to a body part of the worker and display the corrected skeletal information of the worker on the screen. Here, the skeletal structure correction apparatus may display the correction result of the skeletal information of the worker through a tool for assessing the posture of the worker through ergonomics.

The components described in the embodiments may be implemented by hardware components including, for example, at least one digital signal processor (DSP), a processor, a controller, an application-specific integrated circuit (ASIC), a programmable logic element, such as a field programmable gate array (FPGA), other electronic devices, or combinations thereof. At least some of the functions or the processes described in the embodiments may be implemented by software, and the software may be recorded on a recording medium. The components, the functions, and the processes described in the embodiments may be implemented by a combination of hardware and software.

The method according to embodiments may be written in a computer-executable program and may be implemented as various recording media such as magnetic storage media, optical reading media, or digital storage media.

Various techniques described herein may be implemented in digital electronic circuitry, computer hardware, firmware, software, or combinations thereof. The implementations may be achieved as a computer program product, i.e., a computer program tangibly embodied in an information carrier, e.g., in a machine-readable storage device (for example, a computer-readable medium) or in a propagated signal, for processing by, or to control an operation of, a data processing apparatus, e.g., a programmable processor, a computer, or multiple computers. A computer program, such as the computer program(s) described above, may be written in any form of a programming language, including compiled or interpreted languages, and may be deployed in any form, including as a stand-alone program or as a module, a component, a subroutine, or other units suitable for use in a computing environment. A computer program may be deployed to be processed on one computer or multiple computers at one site or distributed across multiple sites and interconnected by a communication network.

Processors suitable for processing of a computer program include, by way of example, both general and special purpose microprocessors, and any one or more processors of any kind of digital computer. Generally, a processor will receive instructions and data from a read-only memory or a random-access memory, or both. Elements of a computer may include at least one processor for executing instructions and one or more memory devices for storing instructions and data. Generally, a computer also may include, or be operatively coupled to receive data from or transfer data to, or both, one or more mass storage devices for storing data, e.g., magnetic, magneto-optical disks, or optical disks. Examples of information carriers suitable for embodying computer program instructions and data include semiconductive wire memory devices, e.g., magnetic media such as hard disks, floppy disks, and magnetic tape, optical media such as compact disk read only memory (CD-ROM) or digital video disks (DVDs), magneto-optical media such as floptical disks, read-only memory (ROM), random-access memory (RAM), flash memory, erasable programmable ROM (EPROM), or electrically erasable programmable ROM (EEPROM). The processor and the memory may be supplemented by, or incorporated in special purpose logic circuitry.

In addition, non-transitory computer-readable media may be any available media that may be accessed by a computer and may include both computer storage media and transmission media.

Although the present specification includes details of a plurality of specific embodiments, the details should not be construed as limiting any invention or a scope that can be claimed, but rather should be construed as being descriptions of features that may be peculiar to specific embodiments of specific inventions. Specific features described in the present specification in the context of individual embodiments may be combined and implemented in a single embodiment. On the contrary, various features described in the context of a single embodiment may be implemented in a plurality of embodiments individually or in any appropriate sub-combination. Furthermore, although features may operate in a specific combination and may be initially depicted as being claimed, one or more features of a claimed combination may be excluded from the combination in some cases, and the claimed combination may be changed into a sub-combination or a modification of the sub-combination.

Likewise, although operations are depicted in a specific order in the drawings, it should not be understood that the operations must be performed in the depicted specific order or sequential order or all the shown operations must be performed in order to obtain a preferred result. In specific cases, multitasking and parallel processing may be advantageous. In addition, it should not be understood that the separation of various device components of the aforementioned embodiments is required for all the embodiments, and it should be understood that the aforementioned program components and apparatuses may be integrated into a single software product or packaged into multiple software products.

The embodiments disclosed in the present specification and the drawings are intended merely to present specific examples in order to aid in understanding of the present disclosure, but are not intended to limit the scope of the present disclosure. It will be apparent to those skilled in the art that various modifications based on the technical spirit of the present disclosure, as well as the disclosed embodiments, can be made.

What is claimed is:

1. A skeletal structure correction method comprising:
collecting an operation image of a worker, divided into operation units;
determining skeletal information of the worker by considering a capturing angle of the collected operation image;
determining an action level of an operation posture of the worker for each operation unit based on the determined skeletal information of the worker; and
correcting the skeletal information of the worker, corresponding to the determined action level.

2. The skeletal structure correction method of claim 1, wherein the collecting of the operation image of the worker comprises collecting an operation image including the whole body of the worker or collecting a plurality of operation images of a same operation captured in different angles, through an image device.

3. The skeletal structure correction method of claim 1, wherein the collecting of the operation image of the worker comprises collecting an operation image divided into at least one operation unit of a waist, upper limbs, and lower limbs, according to an operation motion of the worker.

4. The skeletal structure correction method of claim 1, wherein the determining of the skeletal information of the worker comprises determining the skeletal information of the worker by using at least one operation image of a waist, upper limbs, and lower limbs according to an operation posture of the worker.

5. The skeletal structure correction method of claim 1, wherein the determining of the skeletal information of the worker comprises:
estimating a body part of the worker corresponding to the operation unit;
determining a location of a joint of the worker by considering a feature of a musculoskeletal system according to the body part of the worker; and
determining skeletal information of a worker corresponding to the location of the joint of the worker.

6. The skeletal structure correction method of claim 5, wherein the determining of the action level comprises determining an action level indicating a risk level of an operation for each operation posture of the worker, by considering a motion direction of the joint according to the location of the joint.

7. The skeletal structure correction method of claim 1, wherein the correcting of the skeletal information of the worker comprises correcting the skeletal information of the worker to improve the operation posture of the worker corresponding to the action level.

8. A skeletal structure correction method comprising:
displaying operation images including skeletal information about an operation posture of a worker working in a workplace;
activating and displaying at least one operation image including an operation posture corresponding to a predetermined threshold among the operation images; and
displaying a correction result of the skeletal information of the worker within the activated and displayed operation image on a screen, by considering an angle of a joint according to the operation posture.

9. The skeletal structure correction method of claim 8, wherein the displaying of the operation images comprises displaying, on the screen, an operation image divided into at least one operation unit of a waist, upper limbs, and lower limbs, according to an operation motion of the worker.

10. The skeletal structure correction method of claim 8, wherein the activating and displaying of the operation image comprises:
determining whether each angle of the joint according to the operation posture of the worker exceeds a predetermined threshold in the operation images; and
activating and displaying at least one operation image exceeding the predetermined threshold.

11. The skeletal structure correction method of claim 8, wherein the displaying of the correction result of the skeletal information on the screen comprises:
correcting the skeletal information of the worker to improve the operation posture of the worker by considering a feature of a musculoskeletal system, according to a body part of the worker; and
displaying the corrected skeletal information of the worker on the screen.

12. A skeletal structure correction apparatus comprising a processor, wherein the processor is configured to:
collect an operation image of a worker, divided into operation units;
determine skeletal information of the worker by considering a capturing angle of the collected operation image;
determine an action level of an operation posture of the worker for each operation unit based on the determined skeletal information of the worker; and
correct the skeletal information of the worker, corresponding to the determined action level.

13. The skeletal structure correction apparatus of claim 12, wherein the processor is configured to:
estimate a body part of the worker corresponding to the operation unit;
determine a location of a joint of the worker by considering a feature of a musculoskeletal system according to the body part of the worker; and
determine skeletal information of a worker corresponding to the location of the joint of the worker.

14. The skeletal structure correction apparatus of claim 13, wherein the processor is configured to determine an action level indicating a risk level of an operation for each operation posture of the worker, by considering a motion direction of the joint according to the location of the joint.

15. The skeletal structure correction apparatus of claim 12, wherein the processor is configured to determine the action level by applying an angle according to a location of a joint of the worker within the operation image.

16. The skeletal structure correction apparatus of claim 12, wherein the processor is configured to correct the skeletal information of the worker to improve the operation posture of the worker corresponding to the action level.
